(19) United States
(12) Patent Application Publication
(10) Pub. No.: US 2015/0322397 A1
(43) Pub. Date: Nov. 12, 2015

(54) Bioreactor Cartridge and System

(71) Applicant: California Stem Cell, Inc., Irvine, CA (US)

(72) Inventors: Andrew Cornforth, Mission Viejo, CA (US); Gabriel Nistor, Laguna Niquel, CA (US)

(21) Appl. No.: 14/410,085

(22) PCT Filed: Jun. 18, 2013
(86) PCT No.: PCT/US13/46391

§ 371 Date: Dec. 21, 2014

Related U.S. Application Data
(60) Provisional application No. 61/808,954, filed on Apr. 5, 2013.
(60) Provisional application No. 61/662,859, filed on Jun. 21, 2012.

(51) Int. Cl. C12M 3/00; C12M 1/00; C12M 1/12

(52) U.S. Cl. CPC C12M 23/42; C12M 25/10; C12M 29/10; C12M 25/14

(57) ABSTRACT

A bioreactor with a removable reactor core having internal growth chambers, a first end with an inlet upstream from said core; a second end downstream with an outlet from said core; and, a pumping means to provide media flow, is disclosed.

### CROSS-REFERENCE TO RELATED APPLICATIONS

This application claims the full Paris Convention priority to, and benefit of U.S. provisional applications 61/662,859 filed Jun. 21, 2012, and 61/808,954, filed Apr. 5, 2013, the contents of which are incorporated by this reference as if fully set forth herein in their entirety.

### FIELD

The present disclosure relates to fast growth bioreactors.

### BACKGROUND

Traditional commercial bioreactors are geared to high cell density and large amount of the biomass growth. Some examples are found at the following internet locations:

http://pbsbiotech.com/category/press-release/bioreactor/.

http://www.ecomagination.com/portfolio/wave-bioreactor-for-biotheraputics-production.

http://www.celltainer.com/home.html.

http://www.greinerbioone.com/UserFiles/File/IVSSbrochure.pdf.

http://www.accentia.net/media/docs/AutovaxIDBrochure.pdf.

http://www.fibercellsystems.com/products_cartridqes.htm.

http://www.applikon-biotechnology.us/index.php?option=com_content&view=category&id=42&layout=blog&Itemid=321.

http://www.ncbi.nlm.nih.gov/pubmed/16929403.

http://sim.confex.com/sim/2009/techprogram/P11876.HTM’.

http://www.dasgip.com/media/content/catalog/pdf/DASGIP_E-Flyer_Products_DASbox_en.pdf.

http://www.millipore.com/catalogue/module/c84539.

http://www.fernandocamacho.com/publicaciones/Development%20%of%20a%20Prototype%20Hollow%20Fibre%20Bioreactor%20System%20-%20Master20Thesis.pdf

http://www.bioprocessintl.com/multimedia/archive/00079/BPI_A_{—}090709AR14_O_{—}79769a.pdf.

http://www.bioprocessintl.com/multimedia/archive/00078/BPI_A_{—}090702SUPAR04_{—}78862a.pdf.

http://www.faqs.org/patents/app/20080299539.

http://www.faqs.org/patents/app/20110136225.

http://www.faqs.org/patents/app/20090148941.

http://www.faqs.org/patents/app/20090053762.

http://www.visiongain.com/Report/805/Single-Use-Bioreactors-for-Pharma-World-Market-2012-2022.

### DESCRIPTION

As used herein, including the appended claims, the singular forms of words such as “a,” “an,” and “the” include their corresponding plural references unless the context clearly dictates otherwise. All references cited herein are incorporated by reference to the same extent as if each individual publication, patent, published patent application, and sequence listing, as well as figures and drawings in said publications and patent documents, was specifically and individually indicated to be incorporated by reference.

Some aspects of the exemplary implementations disclosed herein relate to individualized cell expansion when multiple cell sources cannot be combined and must be grown in parallel and closed system. The bioreactor can be sized for cell quantities that are not practical to grow in flasks by traditional methods, for example 500×10⁶ in individual batches.

Some aspects of the exemplary implementations disclosed herein relate to cell proliferation with low cost, minimal operator intervention, based on single use, disposable cartridges. In some aspects cartridges can be installed in an array of devices, each monitored and controlled separately.

The devices and system, addresses and can, in some instances, reduce contaminations thereby being the compliance with the good manufacturing practices required for biological and pharmaceutical drugs manufacturing for human use.

Growth chambers disclosed herein are pipes which may be circular, geometric or complex in cross section and such tubes or pipes have small diameters and are substantially longer than the diameter. The fluid dynamics in exemplar systems can be approximated with the Hagen-Poiseuille equation assuming that the flow is laminar, viscous and incompressible and there is no acceleration of the liquid in the pipe. The total flow is limited by the maximum fluid velocity which does not create Reynold numbers near turbulent flow. Therefore a range of diameters can be used to constrain the system for the required total cell number and maximum fluid velocity. The growth chambers may have diameters generally in the range of about 0.1 mm to about 2 mm, preferably in the range of about 0.5 mm to about 1.5 mm and most preferably in the range of about 0.9 mm to about 1.1 mm.

The generally tubular growth chambers are preferably impermeable to diffusion or movement of materials or fluids through the sidewalls including gases i.e. no exchange exists between the intra and extra capillary compartment.

The nutriments and gas exchange for the cells are provided via media circulated one way through the growth chambers. In traditional bioreactor systems, the media is recirculated until depletion. Recirculation may compromise the sterility and identity of the cells in the cartridge units; it can also increase the complexity and maintenance requirements.

A bioreactor comprising a reactor core having internal growth chambers, a first end with an inlet upstream from said core; a second end downstream with an outlet from said core; and, a pumping means to provide media flow.

A bioreactor system comprising an array of reactor cores having internal growth chambers, a first end with an inlet and a second end with an outlet; a pumping means to provide media flow; and a common fresh media supply.

A bioreactor comprising a reactor core having internal growth chambers, a first end with an inlet upstream from said core; a second end downstream with an outlet from said core; and, a pumping means to provide media flow.

A bioreactor further comprising at least one of flow conditioning grid, a means for heat transfer to said media upstream from said core, a means to oxygenate the media upstream from said core and at least one sensor upstream and/or downstream from said core.

A bioreactor system comprising an array of reactor cores having internal growth chambers, a first end with an inlet and a second end with an outlet; a pumping means to provide media flow; and a common fresh media supply.

Some aspects of the exemplary implementations disclosed herein are a biological growth device having a reactor with a growth chamber unit “GCU” having an inlet cap, flow conditioning membrane, harvesting cap, closed flow channels forming an array, and, whereby a matrix of said closed flow channels is constructed via affixing layers having open flow channels. In some instances, the flow channels are generally square or ovoid. In some instances, the flow channels are formed between a bottom and top with vertical sides. In some instances, the junction between the vertical sides (7) and top has a radius.

Some aspects of the exemplary implementations disclosed herein are a biological growth device having a reactor with a growth chamber unit “GCU” having an inlet cap, flow conditioning membrane, harvesting cap, closed flow channels forming an array and, whereby a matrix of said closed flow channels is constructed via affixing layers having open flow channel, and a digital memory is attached to the GCU.

Some aspects of the exemplary implementations disclosed herein is a layer of an array comprising a series of open flow guides in a stackable layer; and, wherein stacking said layers closes off the open flow guides.

Some aspects of the exemplary implementations disclosed herein is a method of growth in a biological growth system, the method comprising controlling flow rates of media in the closed flow channel of internal growth chambers (IGC) of a bioreactor which limit shear stress in the flow channels to reduce shear stress damage on cells being grown therein. In some instance the shear stress produced by media flow rates in the closed flow channels is limited to less than about 5 Pa. In some instances the shear stress produced by media flow in the flow channels limited to less than 30 minutes at 7.6 Pa via flow rate of media.

Some aspects of the exemplary implementations disclosed herein is a method of growth in a biological growth system, the method comprising controlling flow rates of media in the closed flow channel of internal growth chambers (IGC) of a bioreactor which limit shear stress in the flow channels to reduce shear stress damage on cells being grown therein and providing for the nutriment and oxygenation requirement of the cells.

Some aspects of the exemplary implementations disclosed herein is a method of growth in a biological growth system, the method comprising controlling flow rates of media in the closed flow channel of internal growth chambers (IGC) of a bioreactor which limit shear stress in the flow channels to reduce shear stress damage on cells being grown therein and limiting nutritional or oxygenation gradients along the length of the IGC so that a maximal intermittent flow can be provided. In some instances the media contained in biological growth system from inlet to outlet contains about to 200 μmol O₂ with a gradient of less than 30%. In some instances the media contained in biological growth system from inlet to outlet contains about to 200 μmol O₂ with a gradient of less than 30%.

Some aspects of the exemplary implementations disclosed herein is a biological growth system, having a plurality of reactors each with a growth chamber unit, an inlet, an outlet, media delivery upstream from the inlet; one or more pumps upstream from the inlet; oxygen delivery upstream from the inlet; at least one of dissolved O₂, temperature and pH sensors upstream of the inlet; and, one or more output sensors for measuring dissolved O₂ and pH downstream of the inlet.

Some aspects of the exemplary implementations disclosed herein is a biological growth system, having a plurality of reactors each with a growth chamber unit, an inlet, an outlet, media delivery upstream from the inlet; one or more pumps upstream from the inlet; oxygen delivery upstream from the inlet; at least one of dissolved O₂, temperature and pH sensors upstream of the inlet; one or more output sensors for measuring dissolved O₂ and pH downstream of the inlet, and monitoring and control of the system. In some instances, the monitoring and control is at least one of input sensors, output sensors, dissolved O2, pH, media, flow rate of media, pumps, oxygen delivery, dissolved O2, and temperature.

### DEFINITIONS

A bioreactor may refer to any manufactured or engineered device or system that supports a biologically active environment. In one case, a bioreactor is a vessel in which a chemical process is carried out which involves organisms or biochemically active substances derived from such organisms. This process can either be aerobic or anaerobic. These bioreactors are commonly cylindrical. A bioreactor may also refer to a device or system meant to grow cells or tissues in the context of cell culture. These devices are being developed for use in tissue engineering or biochemical engineering.

### FIGURES

FIG. 1 shows an exemplary of a bioreactor cross section;

FIG. 2 shows a Bioreactor in a longitudinal section. The flow conditioning grid is placed in front of the entrance after the inlet port. The device is ported by a standard ¼″ inlet and outlet entering from side. The flow metering is done by measuring the pressure loss across the growth area.

FIG. 3 shows a Bioreactor in a longitudinal section, with Luer-lock attached cell reservoir for direct centrifugation. The ports are attached to the cartridge body with Luer-locks.

FIG. 4 shows an array of small Bioreactors/reactor cores in a system configuration.

FIGS. 5A-5D shows a Bioreactor in a longitudinal section, an exploded view, cutaway and an end view.

FIGS. 6A and 6B show a Bioreactor in an exploded view.

FIG. 7 shows a Bioreactor in an exploded view.

All callouts in the attached figures and within tables are hereby incorporated by this reference as if fully set forth herein.

It should be appreciated that, for simplicity and clarity of illustration, elements shown in the figures have not necessarily been drawn to scale. For example, the dimensions of some of the elements are exaggerated, relative to each other, for clarity. Further, where considered appropriate, reference numerals have been repeated among the Figures to indicate corresponding elements. While the specification concludes with claims defining the features of the present disclosure that are regarded as novel, it is believed that the present disclosure's teachings will be better understood from a consideration of the following description in conjunction with the figures and tables in which like reference numerals are carried forward.

### Further Descriptions

Persons of ordinary skill in the art will recognize that the disclosure herein references some operations that are performed by a computer system. Operations which are sometimes referred to as being computer-executed. It will be appreciated that such operations are symbolically represented to include the manipulation by a processor, such as a CPU, with electrical signals representing data bits and the maintenance of data bits at memory locations, such as in system memory, as well as other processing of signals. Memory locations wherein data bits are maintained are physical locations that have particular electrical, magnetic, optical, or organic properties corresponding to the data bits.

When implemented in software, elements disclosed herein are aspects of some of the code segments to perform necessary tasks. The code segments can be stored in a non-transitory processor readable medium, which may include any medium that can store information. Examples of the non-transitory processor readable mediums include an electronic circuit, a semiconductor memory device, a read-only memory (ROM), a flash memory or other non-volatile memory, an optical disk, a hard disk, etc. The term module may refer to a software-only implementation, a hardware-only implementation, or any combination thereof. Moreover, the term servers may both refer to the physical servers on which an application may be executed in whole or in part.

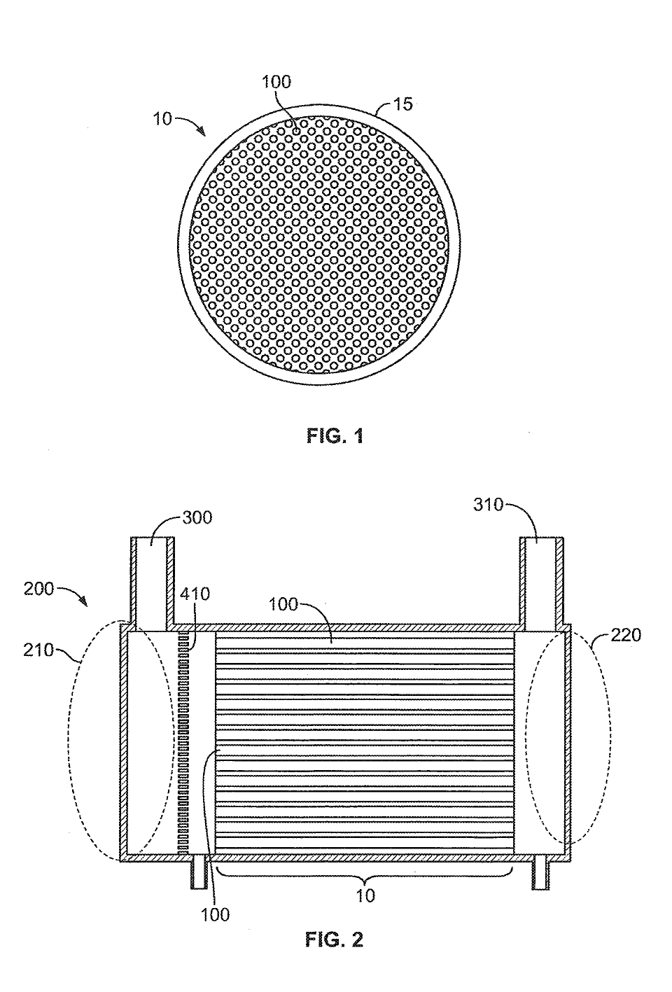

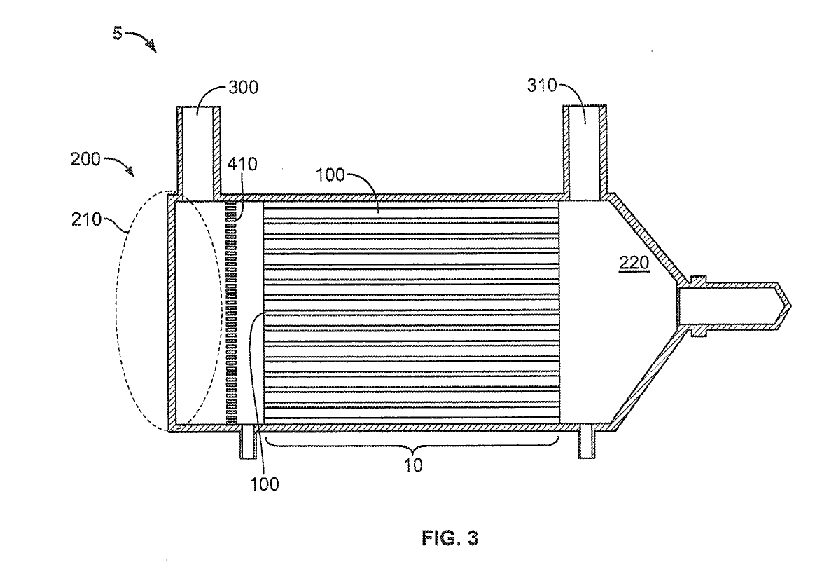

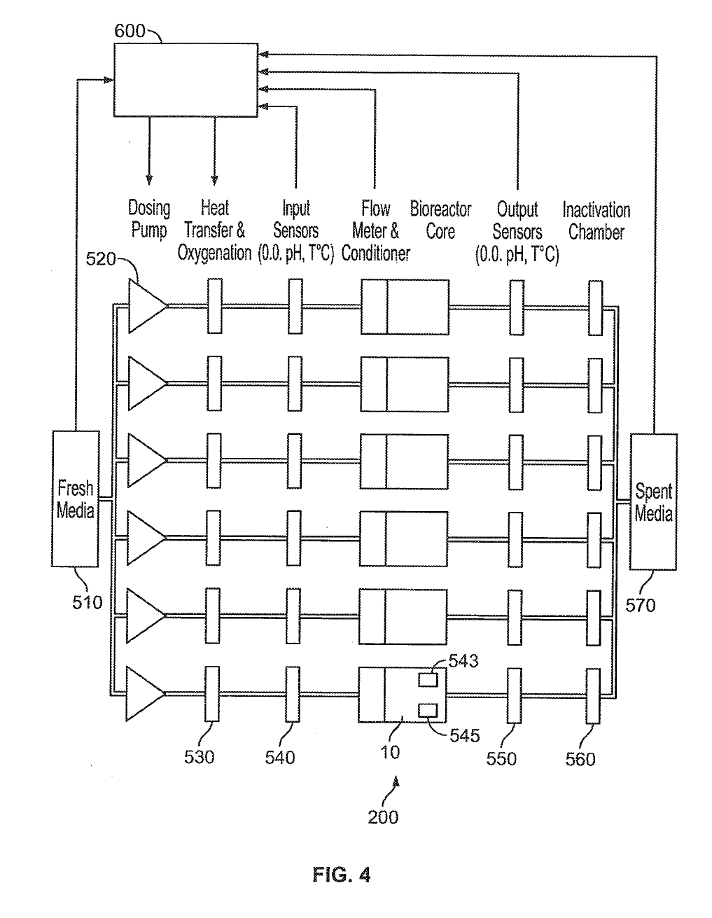

As illustrated in FIGS. 1-4 a bioreactor unit, which also may act as a replace cartridge is preferably constructed of a plastic material, for example, polystyrene or other surfaces that can be modified for optimal cell attachment. The growth chamber unit “GCU” 10 has an outer annular wall 15 and internal growth chambers “IGC” 100. A reactor core 200 with a GCU 10 having, an inlet 300 and an outlet 310. In some exemplars the reactor core includes an inlet cover or cap 210 having the inlet 300 and an outlet cover or cap 220 having the outlet 310. An exact count of polystyrene jacketed poly-methyl methacrylate fibers (PMMA) are placed in a cylinder with a potting material. After the potting material is cured, this version of a GCU 10 is cut in desired lengths and the PMMA core is chemically etched, leaving behind a series of parallel growth chambers within the inner diameter of initial PMMA core. Other materials can replace the polystyrene or the PMMA in a similar process. In some instances, the inner surfaces of the IGC 100 can be coated with collagen or fibronectin or other substrate to promote cell growth. The device should be kept horizontal and non-rotating to promote cell adhesion and cell-to-cell interaction and the lower portion of the IGC 100 is filled with cells.

Two ported end caps are formed as part of, or attached at each end of the GCU 10, forming the reactor core 200. The ports are preferably recessed in the cap material and entering at an angle (15°-60°) designed with Luer-lock coupling for easy manipulation and sterility preservation. The angled port geometry ensures the mixing of the fresh media and homogenous distribution before entrance.

The exit cap 220 may be provided with a reservoir 430 of about 10 mm width and 20 mm length for cell collection connected to the cartridge body with Luer-lock. Centrifuge bucket inserts that can accommodate the described geometry can be made for easy device centrifugation for cell collection.

The system is dimensioned as desired for large growth surface while maintaining a flow which satisfies the cell requirements with a minimal shear stress.

To improve distribution of the liquid at the entrance to the GCU 10 a flow conditioning grid or screen 410 is used. The flow conditioning grid insures the uniform flow distribution across the entire section of the cartridge as the inlet port is placed in front of the conditioning grid. That forms an apparent mixing chamber with the head pressure distributed uniformly on the grid surface.

In the exemplified geometry in the present disclosure, the maximum flow rate through the flow conditioning grid does not reach Reynold number for turbulent flow. The pressure drop across the grid in the anticipated maximum flow is in the range of 0.1-0.2 cm water, obtained from Bernoulli's equation for orifice flow.

Before and after capillary section, two small metering ports are placed to estimate the pressure drop. Knowing the pressure drop, the liquid flow, speed or capillary diameter can be calculated. The same ports can be used for sampling or for cell manipulations.

The inlet is connected to a media reservoir which can be kept refrigerated. The connector tubing made of silicone rubber (ex. Silastic) is coiled around a heat transfer unit. The tubing length is calculated to accomplish the proper oxygenation and heat transfer for maximum media flow.

At the bioreactor exit a neutralizing device can be placed in the circuit to prevent back-contamination of the system. Such device can be a heating element which can warm the output flow to 80-90 C. Alternatively a UV light source or a chemical solution can be used for the same purpose. The spent media is collected in a reservoir which can be removed and disposed as biological waste.

When utilizing the system 500 and reactor device(s), the bioreactor is initially inoculated with a minimal number of cells suspended in a defined media volume. The device is maintained horizontally until the cells attach to the substrate inside of the capillaries. Due to the circular geometry of the tubular elements, the cells are sedimented in a smaller area at seeding (approximately lower ⅓ of the capillary inner surface) critical for the threshold density that promotes cell growth. The substrate consists of a biological active compound recognized by the cell surface deposited on the inner surface of the capillaries. Examples of substrates are proteins, laminin, gelatin, collagen, fibronectin; proteoglycans, silanes with active terminations; combinations or constructs of the exemplified individual compounds in various proportions.

The system 500 is continuously or intermittently perfused with a determined volume of media 510 ranging from a minimum that can be delivered by pumps 520 to the requirements by the maximum cell number that can be achieved by design.

One aspect of the exemplars disclosed herein are flow rates through the devices, which are calculated with the following criteria:

Provide for at least one of the nutriment and oxygenation requirement; a developed laminar flow; limit damaging of the cells caused by media flow (shear stress); limit nutritional or oxygenation gradients along the length of the IGC so that a maximal intermittent flow can be provided.

In the geometry presented in the attached figures, at 0.02 psi pressure drop in the capillaries the flow is about 165 mL/day. At the maximum cell density requiring about 600 mL/day the flow can be achieved with a pressure drop of 0.08 psi with common, low pressure mini-peristaltic pumps. With the flow conditioner designed for minimal pressure drop, a small pump operating at less than 1 psi can satisfy the pressure requirements. At both extremes, the flow in the IGC achieves low speed, laminar flow with no anticipated damage on the cells. By calculating the oxygen requirements the pump doesn't have to work continuously, a stepper motor driven peristaltic pump can be programmed in response to the measured dissolved oxygen level (D.O.) to a ⅔ depletion level.

As the cells expand in a monolayer, they will occupy progressively a portion of the inner surface of the IGC causing a reduction in diameter, but more significant are changes in nutritional perfusion requirements. Hence sensing of the growth before and after media is introduced is utilized. The measured parameters: dissolved oxygen (DO), metabolic by-products (lactic acid), pH, or turbidity can be used to estimate the total cell number and density. Media should be substantially 37 C when entering the reactor core 200, and O₂ saturation should be at a preselected level. When entering the reactor core, a heating means (such as a coil) and O₂ delivery input 530 are placed upstream from the reactor core 200. Dissolved O₂, temperature and pH sensors 540 are also placed upstream of the reactor core 200.

The sensors and software may be provided in an OEM package by third party manufacturer (for example PreSens, Germany—http://www.presens.de/engineering-services/oem-solutions.html). The sensors are connected to a multichannel data acquisition, processed by software with output to control the pumping speed, aerator and alarms. When the captured parameters indicate that the cell population expanded to the required amount, the media is replaced by a proteolithic enzyme and the cells are collected at the output.

In addition to the one or more input sensors 540 one or more output sensors 550 for measuring dissolved O₂ and pH are monitored downstream 585 from the reactor core. The monitoring may include monitoring of one or more of the upstream sensors which monitor media, pumps, oxygen delivery, dissolved O₂, temperature and pH sensors. This monitoring and control can include human interface and computer control. Measurements outside nominal may be used to set and set off alarms or remedial steps.

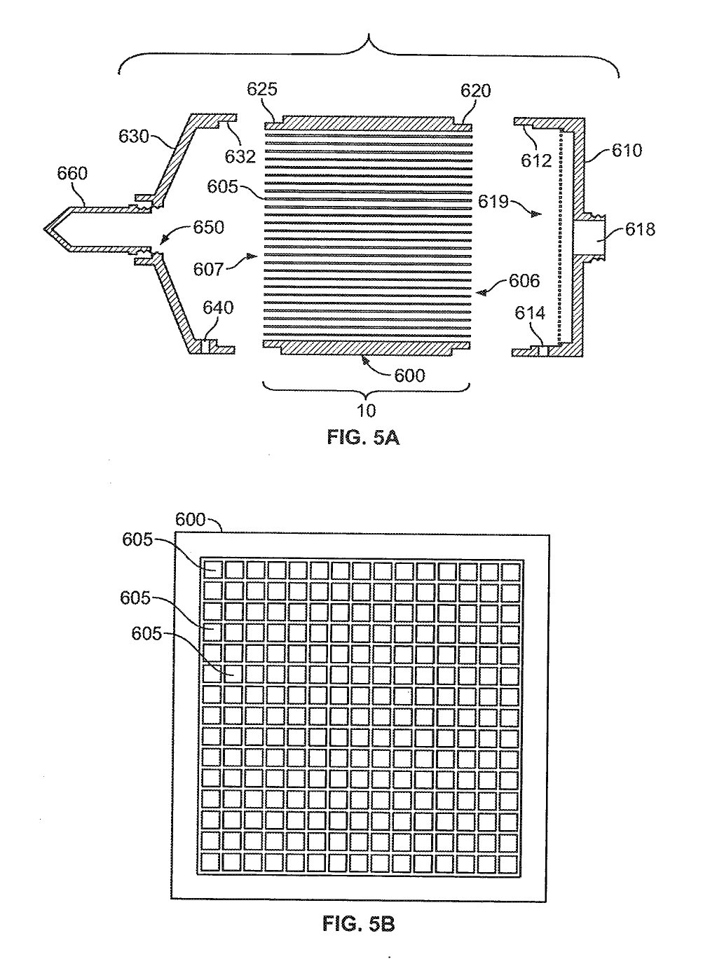

The control hardware and software can be integrated in a disposable chip that is attached to the cartridge 543 or it can be external to the cartridge or a hybrid with components on the cartridge and some external. The control system should contain sufficient memory to store functional parameters sampled at a small time interval for 4-6 weeks, or longer. The memory or a duplicate of the memory 545 may be affixed to the cartridge. That configuration provides for a code to be electronically placed on each cartridge (such as a unique identifier) that can be used by a centralized system. The centralized system (see generally FIG. 4) provides electrical power to multiple cartridges, communicates bi-directionally, can perform calibrations, and can produce reports that are available locally or in a network.

### Example of Bioreactor Calculations:

In the following example we present the parameters of a single use cartridge designed to produce up to 350×10̂6 cells. The system parameters and assumptions are listed in table 1.

TABLE 1
System parameters

| | | |
|---|---|---|
| Capillary inner diameter | 1.5 | mm |
| Capillary length | 6 | cm |
| Capillary count | 541 | each |
| Media viscosity | 0.8 | cp |
| Typical feeding volume | 0.4 | ml/cm² |
| Typical cell density | 250,000 | cells/cm² |
| Head pressure | 0.06 | psi |
| Oxygen consumption | 0.012 | μmol/10⁶ cells/h |
| Typical oxygen solubility | 350 | μmol/L (320-420) |

The following tables (2, 3, 4, 5, 6) calculate the system output and fluid dynamic parameters. The formulae were included in an Excel spreadsheet to allow fine tuning of the parameters.

TABLE 2
System geometry calculations

| | Capillary | | Total | | Growth |
|---|---|---|---|---|---|
| | radius | Capillary | Growth Surface | Equivalent | vol |
| | (cm) | Count | (cm2) | in T150 | (ml) |
| System | 0.075 | 541 | 1528.9 | 10 | 57.33 |
| geometry | | | | | |

TABLE 3
Cell yields

| | Total per device | Total per capillary |
|---|---|---|
| Cell yields | 382,216,500 | 706,500 |

TABLE 4
Oxygen requirements

| | Oxygen | Required | Time to total | Time to ⅔ |
|---|---|---|---|---|
| | content per | O₂ for | O₂ depletion of | O₂ depletion of |
| | growth vol. | total cells | growth volume | growth volume |
| | (μmol) | (μmol/h) | (h) | (h) |
| Oxygen | 20.07 | 4.59 | 4.38 | 2.9 |
| require- | | | | |
| ments | | | | |

TABLE 5
Unit conversions

| Unit conversions | Pressure | Viscosity |
|---|---|---|
| Common units | psi | cp |
| | 0.06 | 0.8 |
| Standard units | N/cm2 | Poise (N*sec/cm2) |
| | 0.041368544 | 0.008 |

TABLE 6
Fluid dynamics calculations. The “targeted flow” value is derived
from the empirical feeding volumes used for equivalent tissue culture flasks.

| | | | | Targeted | Average |
|---|---|---|---|---|---|
| Calculated Capillary Flow from Poiseuille's Equation | | | | Flow | velocity |
| cm3/s (mL/s) | mL/min | mL/h | mL/day | mL/day | cm/sec |
| 0.005790432 | 0.347425928 | 20.84555566 | 500.2933359 | 611.55 | 6.0598E−04 |

TABLE 7
Shear Stress calculation
Shear stress

| (N/cm2) | Dynes/m2 | Pa |
|---|---|---|
| 6.4638E−05 | 6.463834959 | 0.6464 |

The shear stress in wall calculated at maximum liquid velocity in the capillaries is below the values cited in the literature having a damaging effect of the cells.

One of the most important parameters is the oxygenation: The systems disclosed herein must adjust to ensure the required amount for the total number of cells.

The media contained in the device (57 ml) contains enough oxygen to feed the cells for 2.9 hours equivalent to ⅔ depletion (or about to 100 μmol O₂ in the media). At the velocity of 0.06 mm/sec it would take about 2.7 hours for a complete exchange in the capillary. This approach will cause a 350 pmolar oxygenation at the entrance and about 100 pmolar oxygenation at the exit from capillaries. The resulting gradient could be unpredictable on the cell growth and should be avoided or limited.

To reduce or avoid oxygen or nutriment gradient along the capillaries, the system can be programmed based on a micro-batch feeding approach. The entire capillary volume (57 ml) is replaced relatively fast, at a speed which causes non damaging shear stress. Previous studies show a decrease in viability after 30 minutes exposure to 7.6 Pa. A shear stress of about 5 Pa can be obtained by increasing the head pressure from 0.06 psi to about 0.5 psi (10 time increase of the pumping speed) causing a complete media exchange in 33 minutes.

Between the extremes (continuous flow with 2.7 hour exchange and batch feed at 33 minutes total exchange with threshold shear stress) the system can be adjusted for optimal flow and oxygenation. For that purpose, the system sensors output (dissolved oxygen sensors, pH readings) is controlling the peristaltic pump which is a stepper motor (Williamson Manufacturing Ltd). Using the batch feeding approach, the media is allowed to be consumed to an established threshold, for example to 50% of the initial oxygen load or about 2 hours in the exemplified geometry, then replaced over a shorter period of time, 30 minutes, to ensure that the Oxygen concentration will not drop below the minimum threshold. Another advantage of the batch feeding approach is that it allows for system maintenance, such as parts replacement, during the non-feeding periods.

The media composition does not need to be altered as in larger scale bioreactors. The media gassing can be ensured by hollow fiber exchangers or Silastic tubing, however excessive oxygenation requirement is not anticipated at the projected cell densities. The pH is not anticipated to fluctuate as in super high density bioreactor, and the media is not recirculated, therefore no additional pH buffering is required.

The bioreactor core 200 can be removed from the system and cells harvested/collected. For harvesting, the media in the reactor core is replaced by a solution to dissociate the cells, the cartridge removed from the system and the ports secured with sterile Luer-lock caps. The entire device may be centrifuged and the cells collected in a cell reservoir. The cell reservoir 430 is then detached from the device and secure closed with a sterile Luer-lock protective cap.

In other instances the bioreactor core 200 can be removed from the system, securely closed, transported or stored and exposed to ionizing or actinic irradiation in order to inactivate or arrest the cell growth. The reactor core 200 can then be super-seeded after irradiation with another cell type population, for example with dendritic cells (DC). The dendritic cell (DC) suspension can be infused in the reactor core 200 via a metering port. This procedure may be useful in conjunction with personalized medicine to create specific DC.

For a DC application after adding DC allow one hour for cell attachment then the normal feeding is restarted with the media formulated for the DC growth. The reactor core 200 can be maintained in the same circuit until DC harvesting.

For harvesting, the media in the cartridge can be replaced by a solution to dissociate the cells, the cartridge removed from the system and the ports secured with sterile Luer-lock caps. The entire device can be centrifuged and the cells collected in the cell reservoir. The cell reservoir is detached from the cartridge and secure closed with a sterile Luer-lock protective cap.

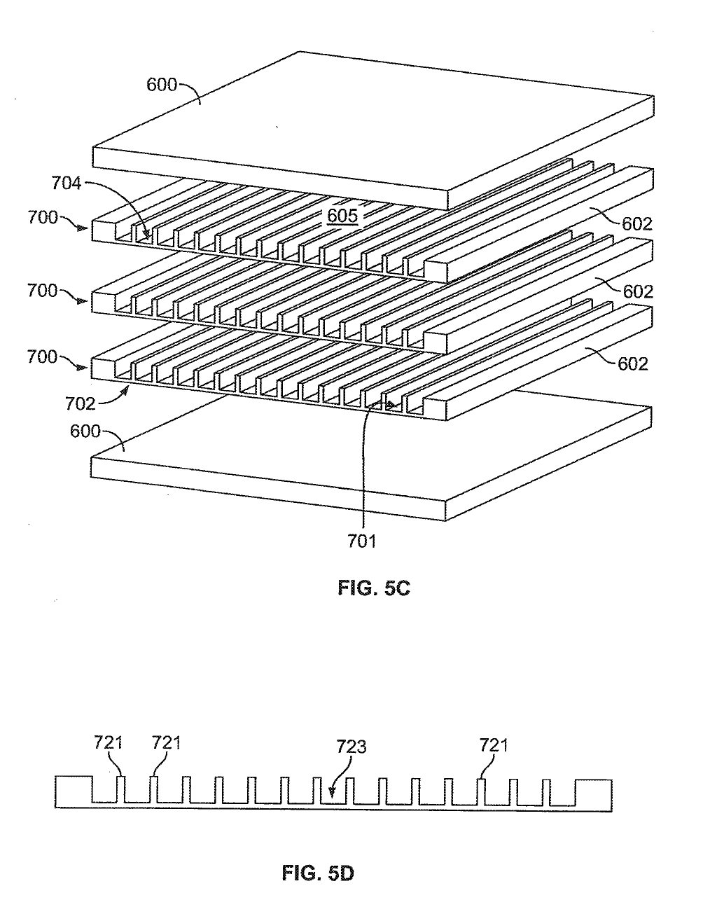

FIGS. 5A-5D show a bioreactor having an array of capillaries, including a GCU 10 within an outer shell 600. The CGUs disclosed herein may also be used with a system as described previously. The array 605, as shown, has an I.D. (internal diameter) of about 2 mm in a parallelepiped. Preferably the array I.D. may be in the range of about 0.5 mm to about 5 mm. The outer shell 600 has a thickness of about 3 mm but may be in the range of about 0.5 to 5 mm to over 10 mm. The wall 721 between sides of array channels maybe in the range of about 0.2 to about 1 mm, and have draft angles to facilitate ease of removal of from a molding machine. However, shear stress on the cells in the GCU should be below the threshold known to damage cells.

The array forms a flow pathway with an inlet side 606 and an outlet side 607. An inlet cap 610 with a first mounting catch 612 mates with a first mounting latch 620 on the outer shell 600. The inlet cap 610 also has a seeding port 614 which may be angled and a Luer-Lock fitted inlet 618. A flow conditioning membrane 619 forms a permeable barrier opposite the inlet 618. The outlet side 607 mates with a harvesting cap 630 via a second mounting catch 632 that fits on a second mounting latch 625 on the outer shell 600. The harvesting cap also has an evacuation port 640 and a Luer-Lock fitted outlet 650 which connects to a vessel 660.

When constructing the array, sandwich layers 700 having a substantially flat bottom 702 and a top 701 having longitudinal flow guides or channels 704 formed by generally vertical walls 721 therein; sandwich layers are stacked and held together via glue, adhesive or sonic welding to form an array 605 of flow guides. Once affixed, the open flow guides 704 are closed, having sides, a top and bottom, forming closed flow guides (not shown) closed flow channel array 605. On two opposing sides of each layer a shell shoulder segment 602 is formed. The shell segment is a thicker region which is a support member of the device when glued to other like regions. At the intersection or junction 723 of the vertical wall and the top 701, the connection may be substantially 90 degrees, or it may have a radius cross-sectional profile. The radius may, in some instances, reduce collection of material at a hard corner (such as a 90 degree area).

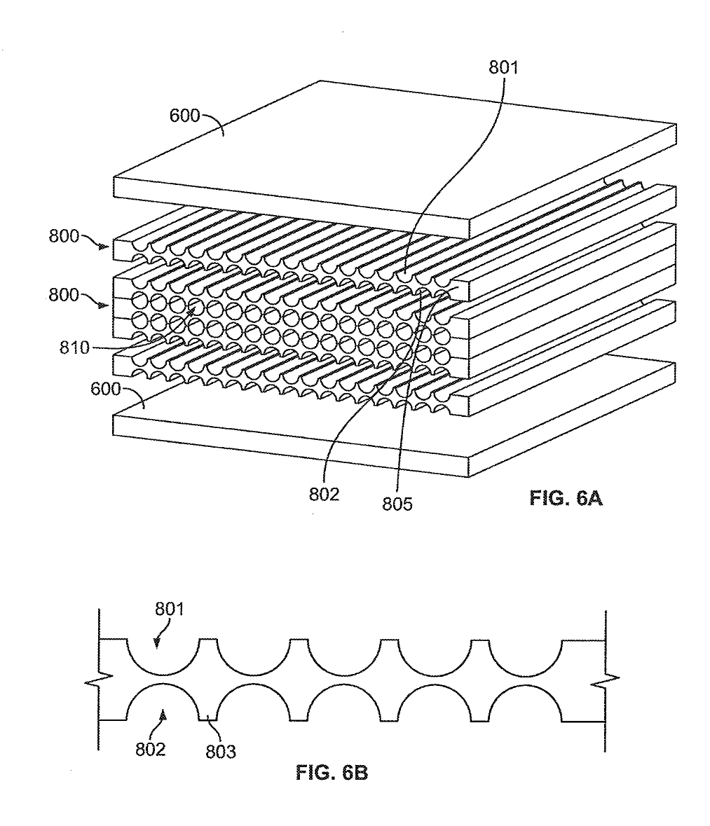

FIGS. 6A and 6B show exemplary implementations of a bioreactor core having a GCU with an array of curved capillaries between an outer shell 600. The CGU disclosed herein may also be used with a system described previously. When constructing the core, sandwich layers 800 are formed with scalloped top sides 801 having a radiussed bottom connecting to sides forming series of semi-circles with a radius in cross-section (see FIG. 6B) and scalloped bottom sides, also with radiussed bottoms 802. A wall 803 separates scalloped channels. The radiussed top sides and the radiussed bottom sides each form an open flow channel. When assembled, sandwich layers are stacked and held together via glue, adhesive or sonic welding to form an array 605 of closed flow guides or flow channels (810)=forming an IGC. That flow channel may have slightly radiussed corners (compared to the exemplary shown in FIG. 5A-5D) or the radius may be as great as semi-circles. The shape of the flow channel is dependent on those radiuses. Using this method a substantially ovoid, radiussed or circular flow channel may be formed.

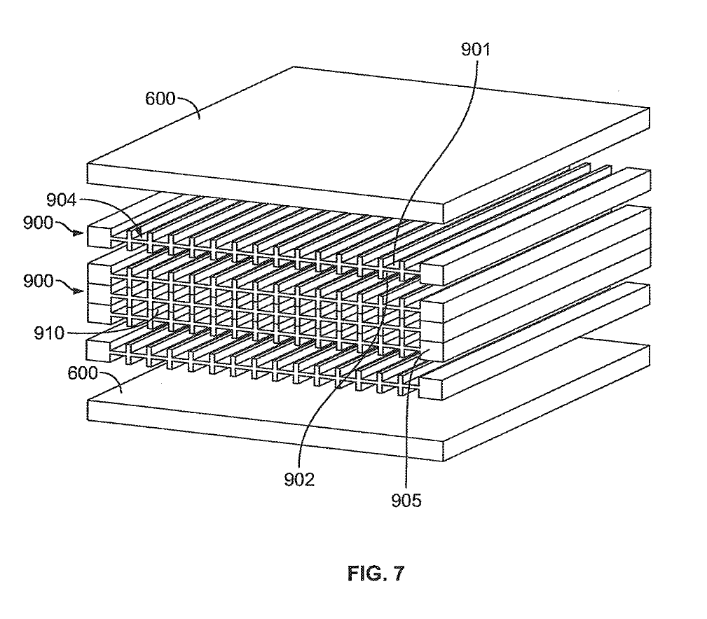

FIG. 7 shows a variation of the square channel array of FIGS. 5A-5D wherein both the top and bottom of the layer have extended walls (721) and mate with another layer forming square channels in a different construction than FIGS. 5A-5D.

FIG. 7 shows an exemplary implementation of a bioreactor core having a GCU and an array of generally rectangular capillaries between an outer shell 600. The CGU disclosed herein may also be used with a system described previously. When constructing the core, sandwich layers 900 are formed with toothed top sides 901 having a generally flat bottom connecting to generally vertical sides forming series of open channels and a series of toothed (or divided) bottom sides 902 with a generally flat roof and generally vertical side walls forming open channels or guides 904. When assembled, sandwich layers are stacked and held together via glue, adhesive or sonic welding to form an array 605 of closed flow guides 910 thereby forming an IGC. The toothed configuration of vertical walls extending from the top and bottom sides of a sandwich layer are shown aligned, to form a flow channel. They may have slightly radiussed corners or junctions 723, or the radius may be as great as semi-circles. The shape of the flow channel is dependent on those radiuses. Using this method, a substantially ovoid, radiussed or circular flow channel may be formed.

Thus, while there have been shown and described and pointed out fundamental novel features of the disclosure as applied to exemplary implementations and/or aspects thereof, it will be understood that various omissions, reconfigurations, substitutions and changes in the form and details of the exemplary implementations, disclosure, and aspects thereof may be made by those skilled in the art without departing from the spirit of the disclosure and/or claims. For example, it is expressly intended that all combinations of those elements and/or method steps which perform substantially the same function in substantially the same way to achieve the same results are within the scope of the disclosure. Moreover, it should be recognized that structures and/or elements and/or method steps shown and/or described in connection with any disclosed form or implementation may be incorporated in any other disclosed or described or suggested form or implementation as a general matter of design choice. It is the intention, therefore, to not limit the scope of the disclosure. All such modifications are intended to be within the scope of the claims appended hereto.

All publications, patents, patent applications and references cited in this specification are herein incorporated by this reference as if fully set forth herein.

The Abstract is provided to comply with 37 CFR §1.72(b) to allow the reader to quickly ascertain the nature and gist of the technical disclosure. The Abstract is submitted with the understanding that it will not be used to interpret or limit the scope or meaning of the claims.

What is claimed is:

1. A biological growth device, comprising:
a reactor (200) having a growth chamber unit (10);
an inlet cap (610);
a flow conditioning membrane (619);
a harvesting cap (630);
closed flow channels (810 and 910) forming an array (605); and,
whereby a matrix (605) of said closed flow channels is constructed via affixing layers having open flow channels (704, 801, 802, 904).

2. The bioreactor of claim 1, wherein the flow channels are generally square.

3. The bioreactor of claim 1, wherein the flow channels are generally ovoid.

4. The bioreactor of claim 2, wherein the flow channels are formed between a bottom (702) and top (701) with vertical sides (721).

5. The bioreactor of claim 4, wherein the junction (723) between the vertical sides (721) and top (701) has a radius.

6. The bioreactor of claim 1, wherein at least one flow channel in the array is selected from the group consisting of square and generally ovoid.

7. The bioreactor of claim 6 further comprising a digital memory (545) attached to the growth chamber unit.

8. The bioreactor of claim 1, further comprising a removable cell collection container (660).

9. A layer of an array comprising a series of open flow guides (904, 803, 802, 704) in a stackable layer; and, wherein stacking said layers closes off the open flow guides.

10. The layer of claim 9, wherein at least one of the closed off flow channels in a stack of layers is selected from the group consisting of square and ovoid.

11. A method of growth in a biological growth system, the method comprising controlling flow rates of media in the closed flow channel of internal growth chambers (IGC) of a bioreactor, which limit shear stress in the flow channels to reduce shear stress damage on cells being grown therein.

12. The method of claim 11, wherein the shear stress produced by media flow rates in the closed flow channels is limited to less than about 5 Pa.

13. The method of claim 11, wherein the shear stress produced by media flow in the flow channels is limited to less than 30 minutes at about 7.6 Pa via flow rate of media.

14. The method of claim 11, the method further comprising providing for the nutrient and oxygenation requirement of the cells.

15. The method of claim 11, the method further comprising limiting nutritional or oxygenation gradients along the length of the IGC, so that a maximal intermittent flow can be provided.

16. The media of claim 11, wherein the media contained in biological growth system from inlet to outlet contains about 200 μmol O₂ with a gradient of less than about 30%.

17. The media of claim 12, wherein the media contained in biological growth system from inlet to outlet contains about 200 μmol O₂ with a gradient of less than about 30%.

18. The media of claim 13, wherein the media contained in biological growth system from inlet to outlet contains about 200 μmol O₂ with a gradient of less than about 30%.

19. A biological growth system, comprising:
a plurality of reactors (200), each having;
a growth chamber unit (10) with a matrix of flow channels;
an inlet (300);
an outlet (310);
media (510) delivery upstream from the inlet;
one or more pumps (520) upstream from the inlet;
oxygen delivery (530) upstream from the inlet;
at least one of dissolved O₂, temperature and pH sensors (540) upstream of the inlet; and,
one or more output sensors for measuring dissolved O₂ and pH (550) downstream of the inlet.

20. The system of claim 19 further comprising monitoring and control (585) of the system.

21. The system of claim 20 wherein the monitoring and control is of at least one of input sensors, output sensors, dissolved O2, pH, media, flow rate of media, pumps, oxygen delivery, dissolved O2, temperature.

22. The system of claim 19, wherein reactors are removable.

23. The system of claim 21, wherein the media contained in the growth chamber unit contains about 200 μmol O₂ with a gradient of less than about 30%.

24. The system of claim 21, wherein the shear stress produced by media flow in the flow channels is limited by flow rate to less than about 7.6 Pa.
